(19) United States
(12) Patent Application Publication
(10) Pub. No.: US 2006/0051298 A1
(43) Pub. Date: Mar. 9, 2006

(54) Abuse resistent pharmaceutical dosage and method of making same

(76) Inventor: Pieter J. Groenewoud, Powder Springs, GA (US)

Correspondence Address: BRYAN W. BOCKHOP, ESQ., 2375 MOSSY BRANCH DR., SNELLVILLE, GA, 30078, US

(21) Appl. No.: 10/934,223

(22) Filed: Sep. 3, 2004

(51) Int. Cl. A61K 9/44; A61K 9/48; A61K 9/20

(52) U.S. Cl. 424/10.2; 424/464; 424/451

(57) ABSTRACT

A pharmaceutical composition includes a therapeutic amount of an active ingredient and at least one gel-forming granule. The gel-forming granule forms a gel when exposed to an aqueous liquid and is coated with an outer coating that is sufficiently brittle so that when the tablet is crushed, a portion of the outer coating will break open so as to expose the gel-forming granule. The outer coating includes a material that resists dissolution when exposed to gastric fluids.

### BACKGROUND OF THE INVENTION

1. Field of the Invention

The invention relates to pharmaceutical compositions, and more specifically, to a pharmaceutical composition formulated to discourage abuse.

2. Description of the Related Art

Certain pharmaceutical compositions, including those subject to abuse, are available in tablet form. Typical tablets include granules of an active ingredient and one or more excipients that affect the physical properties of the tablet. The active ingredient and the excipients are compacted together to form the tablet. Tablet formulations are often desirable because they may be manufactured at a relatively high output with a relatively low manufacturing cost.

Certain compositions, such as narcotics and stimulants, are subject to abuse. Abusers often crush tablets of such compositions and then add water or vinegar to the resultant powder to leach out the active ingredient from the tablet. In this way, abusers are able to combine the active ingredient from several tablets to make a larger dose of the active ingredient. Once this is done, the abuser often heats the active ingredient to melt it and draws it into a syringe for subsequent injection.

One method of reducing abuse of narcotics includes adding coated antagonist particles to a narcotic formulation. If the particles are crushed, the antagonist is released and either the effect of the narcotic is neutralized by the antagonist or the abuser experiences an adverse reaction to the narcotic as a result of the antagonist. This method, however, requires greater regulatory involvement to ensure the safety and effectiveness of the resulting composition.

Therefore, there is a need for a tablet pharmaceutical formulation that inhibits abuse.

There is also a need for a tablet pharmaceutical formulation that reduces the ability of abusers to draw active ingredients leached from the tablet into a syringe.

### SUMMARY OF THE INVENTION

In one aspect, the invention is a pharmaceutical composition that includes a therapeutic amount of an active ingredient and at least one gel-forming granule. The gel-forming granule forms a gel when exposed to an aqueous liquid and may be coated with an outer coating that is sufficiently brittle so that when the tablet is crushed, a portion of the outer coating will break open so as to expose the gel-forming granule. The outer coating includes a material that resists dissolution when exposed to gastric fluids.

In another aspect, the invention is a pharmaceutical tablet that includes a therapeutic amount of an active ingredient and at least one gel-forming granule. The gel-forming granule forms a gel when exposed to an aqueous liquid and is coated with an outer coating that is sufficiently brittle so that when the tablet is crushed, a portion of the outer coating will break open so as to expose the gel-forming granule. The outer coating includes a material that resists dissolution when exposed to gastric fluids. The active ingredient, the gel-forming granule and the outer coating are compacted into a tablet form.

In another aspect, the invention is a method of inhibiting abuse of pharmaceutical compositions in which a gel-forming granule is coated with an outer coating. The gel-forming material forms a gel when exposed to an aqueous liquid. The outer coating is sufficiently brittle so that when the tablet is crushed, a portion of the outer coating will break open so as to expose the gel-forming granule. The outer coating includes a material that resists dissolution when exposed to gastric fluids. An active ingredient having a potential for abuse is mixed with the gel-forming granule.

In yet another aspect, the invention is an abuse-resistant pharmaceutical composition that includes a first plurality of granules combined with a second plurality of granules. The first plurality of granules includes an active ingredient in a therapeutic amount. The second plurality of granules includes a composition that interferes with abuse of the active ingredient when the first plurality of granules and the second plurality of granules are crushed.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE DRAWINGS

FIG. 1 is a top perspective view of one illustrative embodiment of the invention.

FIG. 2A is a cross-sectional view of an illustrative embodiment of the invention, taken along line 2-2 in FIG. 1.

FIG. 2B is a cross-sectional view of a second illustrative embodiment of the invention, taken along line 2-2 in FIG. 1.

FIG. 3A is a cross-sectional view of a gel-forming granule, with a dual coating.

FIG. 3B is a cross-sectional view of a gel-forming granule, with a single coating.

FIG. 4 is a cross-sectional view of a coated active ingredient granule.

### DETAILED DESCRIPTION OF THE INVENTION

A preferred embodiment of the invention is now described in detail. Referring to the drawings, like numbers indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.”

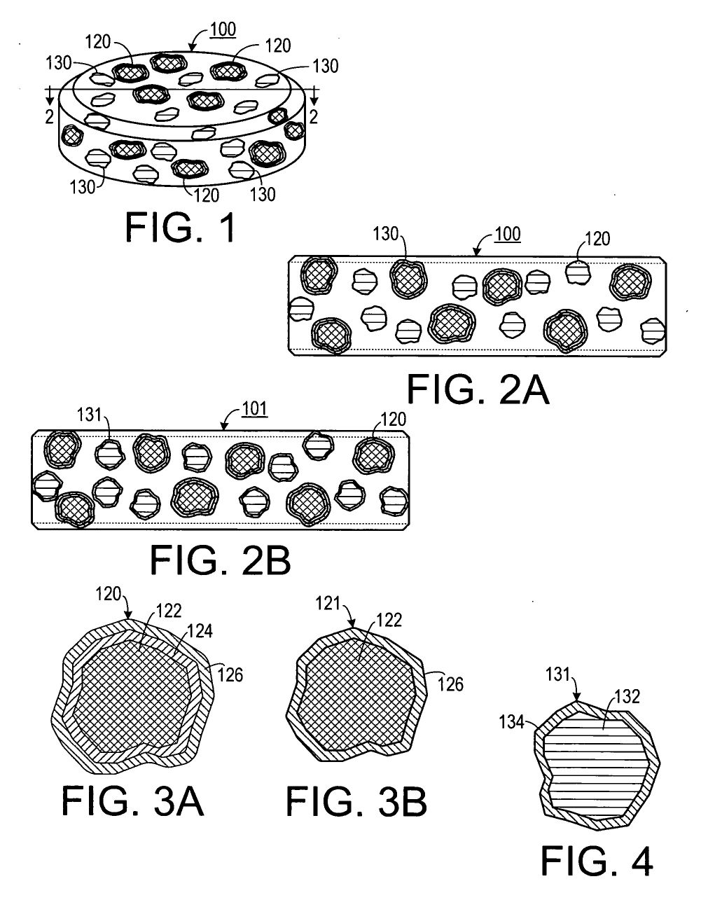

One embodiment of the invention, as shown in FIGS. 1 and 2A, includes a pharmaceutical composition, which may take the form of a tablet 100. The tablet 100 includes a therapeutic amount of active ingredient granules 130 and a plurality of coated gel-forming granules 120, which form a gel when exposed to an aqueous liquid. As shown in FIG. 3A, the coated gel-forming granules 120 are coated with an outer coating 126 that is sufficiently brittle so that when the tablet 100 is crushed, a portion of the outer coating 126 will break open so as to expose the gel-forming granule 122. The outer coating 126 includes a material that resists dissolution when exposed to gastric fluids to prevent the gel-forming granule 122 from forming a gel while the active ingredient is being absorbed in the user's stomach.

The active ingredient granules 130 and the coated gel-forming granules 120 are compacted together so as to form the tablet 100. In alternative embodiments, the active ingredient granules 130 and the coated gel-forming granules 120 may be disposed in a capsule or suspended in a liquid dosage form.

The gel-forming granules 122 may include a material selected from a group including a hydro-colloid; hydroxyl propoxy methyl cellulose; methylcellulose; gellan gum; hydroxyl methyl cellulose; carbomer; carboxy methylcellulose; alginic acid, carrageenan, a eudragit L-type polymethacrylate, a eudragit S-type polymethacrylate, and combinations thereof. As will be readily appreciated by those of skill in the pharmaceutical art, many other commonly known gel-forming materials may be employed as the gel-forming granules 122 without departing from the scope of the invention. The outer coating 126, which may include an enteric coating, may include a material selected from a group including polymethacrylate; cellulose acetate; ethyl cellulose, and combinations thereof. As will be readily appreciated by those of skill in the pharmaceutical art, many other commonly known coatings may be employed as the outer coating 126 without departing from the scope of the invention.

An inner coating 124 may be disposed between the outer coating 126 and the gel-forming granule 122. The inner coating 124 resists dissolution when exposed to intestinal fluids. The inner coating 124 may include a eudragit E-type polymethacrylate, such as ammonio methacrylate copolymer. Thus, if an abuser were to attempt to crush the tablet 100, the coatings 124 and 126 surrounding the gel-forming granules 122 would be broken, and if water or vinegar were applied thereto (for the purpose of leaching the active ingredient) the gel-forming granule 122 would form a gel that would render the active ingredient unusable.

As shown in FIG. 3B, in many applications, only a single coating 126 may be required around the gel-forming granule 122. Such single-coated gel-forming granules 121 may be employed when substantially all of the active ingredient is absorbed in the stomach, or may be employed when a coating 126 may be used that resists both gastric fluids and intestinal fluids.

As shown in FIGS. 2B and 4, the tablet 101 could include coated granules 131 of the active ingredient 132. The coated granules 131 would include a coating 134 disposed about the active ingredient that is soluble in a gastric fluid but that is substantially insoluble in water or a mild acid, such as vinegar. This would ensure that if an abuser were to attempt to dissolve the active ingredient, the coating 134 would have to be crushed along with the coating about gel-forming granules 131. Once the abuser tried to leach the active ingredient with water or vinegar, a gel would form that would bind the active ingredient.

In another embodiment of the invention where it is unlikely that the gel-forming granule, even if fully exposed to gastric fluids 122 will interfere with absorption of the active ingredient 132 in the stomach, even if fully exposed to gastric fluids, either all of the granules may be uncoated or only the active ingredient 132 need be coated. In such an embodiment, the abuser would have to crush the tablet 100 (and the coated active ingredient granules 131 if coating them is necessary) to get at the active ingredient, but would cause a gel to form during the leaching process.

In another embodiment, the invention is an abuse-resistant pharmaceutical composition that includes a first plurality of granules combined with a second plurality of granules. The first plurality of granules includes an active ingredient in a therapeutic amount. The second plurality of granules includes a composition that interferes with abuse of the active ingredient when the first plurality of granules and the second plurality of granules are crushed. When the active ingredient is, for example, an alkaloid, the second plurality of granules could tannic acid that forms an insoluble complex with the active ingredient. The second plurality of granules could also include an emetic, in which case the second plurality of granules are coated with a coating substance that is insoluble in a patient's digestive system. One examples of such a coating includes ethyl cellulose. A double coating may also be used. Such a double coating would include a first coat that is disposed around each of the second plurality of granules and a second coat that is disposed around the first coat. The first coat would include a substance that is resistant to intestinal fluids. The second coat would include a substance that is resistant to gastric fluids. The granules may be compressed into a tablet, placed in a capsule or suspended in a liquid dosage form.

A viscosity enhancer may also be included to increase the viscosity of the pharmaceutical composition, thereby making it harder to draw the pharmaceutical composition into a syringe. The viscosity enhancer could include aluminum stearate, a colloidal silicon dioxide Aerosil; or Cab-C-S/I; Wacker HDK. A temperature sensitive viscosity enhancer, such as a gellan gum (e.g., Kelcogel, Kelcogel F, Kelcogel LT 100 and K7B518, which are available from CP Kelco, 8355 Aero Drive, San Diego, Calif., 92123) could also be used according to the invention. Such a viscosity enhancer becomes highly viscous when heated, such as when an abuser is attempting to melt a pharmaceutical.

Typically, the granules mentioned above are formed through conventional granulation processes well known to the art and the coatings mentioned above are sprayed onto the granules using conventional coating processes (such as those employed in spraying on timed release coatings) well know to the art. Other processes known to the art may be employed for the granulation, coating and compaction of the compositions without departing from the scope of the invention.

A thorough listing of coatings and excipients known to the art may be found in Kibbe, Arthur H., ed.: Handbook of Pharmaceutical Excipients, 3d Edition, 2000, American Pharmaceutical Association, Washington, D.C., which is incorporated herein by reference.

The above described embodiments are given as illustrative examples only. It will be readily appreciated that many deviations may be made from the specific embodiments disclosed in this specification without departing from the invention. Accordingly, the scope of the invention is to be determined by the claims below rather than being limited to the specifically described embodiments above.

What is claimed is:

1. A pharmaceutical composition, comprising:
a. a therapeutic amount of an active ingredient; and
b. at least one gel-forming granule that forms a gel when exposed to an aqueous liquid, the gel-forming granule coated with an outer coating that is sufficiently brittle so that when the tablet is crushed, a portion of the outer coating will break open so as to expose the gel-forming granule, the outer coating including a material that resists dissolution when exposed to gastric fluids.

2. The pharmaceutical composition of claim 1, in which the active ingredient and the gel-forming granule are compacted together so as to form a tablet.

3. The pharmaceutical composition of claim 1, wherein the active ingredient and the gel-forming granule are disposed in a capsule.

4. The pharmaceutical composition of claim 1, wherein the gel-forming granule includes a material selected from a group comprising: a hydro-colloid; hydroxyl propoxy methyl cellulose; methylcellulose; gellan gum; hydroxyl methyl cellulose; carbomer; carboxy methylcellulose; alginic acid, carrageenan, a eudragit L-type polymethacrylate, a eudragit S-type polymethacrylate, and combinations thereof.

5. The pharmaceutical composition of claim 4, wherein the outer coating includes a material selected from a group comprising: polymethacrylate; cellulose acetate; ethyl cellulose, and combinations thereof.

6. The pharmaceutical composition of claim 1, wherein the outer coating comprises an enteric coating.

7. The pharmaceutical composition of claim 1, further comprising an inner coating, disposed between the outer coating and the gel-forming granule, that resists dissolution when exposed to intestinal fluids.

8. The pharmaceutical composition of claim 7, wherein the inner coating comprises a eudragit E-type polymethacrylate.

9. The pharmaceutical composition of claim 8, wherein the eudragit E-type polymethacrylate comprises ammonio methacrylate copolymer.

10. The pharmaceutical composition of claim 1, further comprising a viscosity enhancer that increases the viscosity of the composition when heated above a predetermined temperature.

11. The pharmaceutical composition of claim 1, further comprising an active ingredient coating disposed about the active ingredient that is soluble in a gastric fluid but that is substantially insoluble in water.

12. The pharmaceutical composition of claim 11, wherein the active ingredient coating comprises a eudragit E-type polymethacrylate.

13. The pharmaceutical composition of claim 12, wherein the eudragit E-type polymethacrylate comprises ammonio methacrylate copolymer.

14. A pharmaceutical tablet, comprising:
a. a therapeutic amount of an active ingredient; and
b. at least one gel-forming granule that forms a gel when exposed to an aqueous liquid; and
c. an outer coating that coats the gel-forming granule and that is sufficiently brittle so that when the tablet is crushed, a portion of the outer coating will break open so as to expose the gel-forming granule, the outer coating including a material that resists dissolution when exposed to gastric fluids,
the active ingredient, the gel-forming granule and the outer coating being compacted into a tablet form.

15. The pharmaceutical tablet of claim 14, wherein the gel-forming granule includes a material selected from a group comprising: a hydro-colloid; hydroxyl propoxy methyl cellulose; methylcellulose; gellan gum; hydroxyl methyl cellulose; carbomer; carboxy methylcellulose; alginic acid, carrageenan and combinations thereof.

16. The pharmaceutical tablet of claim 15, wherein the outer coating includes a material selected from a group comprising: polymethacrylate; cellulose acetate; ethyl cellulose, and combinations thereof.

17. The pharmaceutical tablet of claim 14, wherein the outer coating comprises an enteric coating.

18. The pharmaceutical tablet of claim 14, further comprising an inner coating, disposed between the outer coating and the gel-forming granule, that resists dissolution when exposed to intestinal fluids.

19. The pharmaceutical tablet of claim 14, further comprising a viscosity enhancer that increases the viscosity of the composition when heated above a predetermined temperature.

20. The pharmaceutical tablet of claim 14, further comprising a coating disposed about the active ingredient that is soluble in a gastric fluid but that is substantially insoluble in water.

21. A pharmaceutical tablet, comprising:
a. a granule including a therapeutic amount of an active ingredient; and
b. at least one gel-forming granule that forms a gel when exposed to an aqueous liquid.

22. The pharmaceutical tablet of claim 21, further comprising an active ingredient coating disposed about the active ingredient that is soluble in a gastric fluid but that is substantially insoluble in water.

23. The pharmaceutical composition of claim 22, wherein the active ingredient coating comprises a eudragit E-type polymethacrylate.

24. The pharmaceutical composition of claim 23, wherein the eudragit E-type polymethacrylate comprises ammonio methacrylate copolymer.

25. A method of inhibiting abuse of pharmaceutical compositions, comprising the steps of:
a. coating a gel-forming granule, comprising a material that forms a gel when exposed to an aqueous liquid, with an outer coating that is sufficiently brittle so that when the tablet is crushed, a portion of the outer coating will break open so as to expose the gel-forming granule, the outer coating including a material that resists dissolution when exposed to gastric fluids; and
b. mixing an active ingredient having a potential for abuse to the gel-forming granule.

26. The method of claim 25, further comprising the step of compacting the gel-forming granule and the active ingredient to form a tablet.

27. The method of claim 25, further comprising the step of disposing the gel-forming granule and the active ingredient in a capsule.

28. The method of claim 25, wherein the gel-forming granule includes a material selected from a group comprising: a hydro-colloid; hydroxyl propoxy methyl cellulose; methylcellulose; gellan gum; hydroxyl methyl cellulose; carbomer; carboxy methylcellulose; alginic acid, carrageenan and combinations thereof.

29. The method of claim 28, wherein the outer coating includes a material selected from a group comprising: polymethacrylate; cellulose acetate; ethyl cellulose, and combinations thereof.

30. The method of claim 25, wherein the outer coating comprises an enteric coating.

31. The method of claim 25, further comprising an inner coating, disposed between the outer coating and the gel-forming granule, that resists dissolution when exposed to intestinal fluids.

32. The method of claim 25, further comprising a viscosity enhancer that increases the viscosity of the composition when heated above a predetermined temperature.

33. An abuse-resistant pharmaceutical composition, comprising:
a. a first plurality of granules of an active ingredient in a therapeutic amount; and
b. a second plurality of granules, combined with the first plurality of granules, of a composition that interferes with abuse of the active ingredient when the first plurality of granules and the second plurality of granules are crushed.

34. The abuse-resistant pharmaceutical composition of claim 33, wherein the active ingredient comprises an alkaloid and wherein the second plurality of granules comprises tannic acid that forms an insoluble complex with the active ingredient.

35. The abuse-resistant pharmaceutical composition of claim 33, wherein the second plurality of granules comprises an emetic and wherein the second plurality of granules are coated with a coating substance that is insoluble in a patient's digestive system.

36. The abuse-resistant pharmaceutical composition of claim 33, wherein the coating substance comprises ethyl cellulose.

37. The abuse-resistant pharmaceutical composition of claim 33, wherein the coating substance comprises:
a. a first coat, disposed around each of the second plurality of granules, including a substance that is resistant to intestinal fluids; and
b. a second coat, disposed around the first coat, including a substance that is resistant to gastric fluids.

38. The abuse-resistant pharmaceutical composition of claim 33, wherein the second plurality of granules comprises gel-forming granules that form a gel when exposed to an aqueous liquid, the gel-forming granules coated with an outer coating that is sufficiently brittle so that when the tablet is crushed, a portion of the outer coating will break open so as to expose the gel-forming granules, the outer coating including a material that resists dissolution when exposed to gastric fluids.
